(19) United States
(12) Patent Application Publication
(10) Pub. No.: US 2013/0087153 A1
(43) Pub. Date: Apr. 11, 2013

(54) BREATHING SUPPORT DEVICE

(71) Applicant: Seok Soo JUNG, Daegu (KR)

(72) Inventor: Seok Soo JUNG, Daegu (KR)

(21) Appl. No.: 13/267,179

(22) Filed: Oct. 6, 2011

(51) Int. Cl. A62B 7/12; A61M 15/08

(52) U.S. Cl. 128/207.18; 128/200.24

(57) ABSTRACT

The present invention relates to a breathing support device for use in the event of a disaster such as a fire in a building or the like. The breathing support device includes: a connection pipe having any length; an induction pipe connected to one end of the connection pipe; and a supply pipe connected to the other end of the connection pipe, wherein an opening provided in the bottom of the induction pipe is combined with a mesh having a plurality of apertures which is fixed by a fastening piece, and a cover plate is combined to an opening of the supply pipe such that the interior of the supply pipe is sealed by the cover plate and one or more intake/exhaust pipes are combined to the cover plate such that the intake/exhaust pipes communicate to the interior of the supply pipe.

### BACKGROUND OF THE INVENTION

1. Field of the Invention

The present invention relates to a breathing support device, and more particularly, a breathing support device which is capable of providing fresh air for survivors confined in a building where toxic fumes are emitted in a disaster such as a fire.

2. Description of the Related Art

Many life accidents may take happen in the event of fires in big buildings and so on, most of which are attributed to toxic fumes generated in the course of combustion of interior materials in the buildings rather than flames.

### SUMMARY OF THE INVENTION

Accordingly, it is an object of the present invention to provide a breathing support device for protecting persons confined in a building where toxic fumes are emitted in a disaster such as a fire against toxic fumes, which is capable of supplying fresh air into any internal regions of a building to minimize life accidents due to toxic fumes.

To achieve the above object, according to an aspect of the invention, there is provided a breathing support device including: a connection pipe (20) having any length; an induction pipe (30) connected to one end of the connection pipe (20); and a supply pipe (40) connected to the other end of the connection pipe (20), wherein an opening provided in the bottom of the induction pipe (30) is combined with a mesh (31) having a plurality of apertures (31a) which is fixed by a fastening piece (32), and a cover plate (41) is combined to an opening of the supply pipe (40) such that the interior of the supply pipe (40) is sealed by the cover plate (41) and one or more intake/exhaust pipes (42) are combined to the cover plate (41) such that the intake/exhaust pipes (42) communicate to the interior of the supply pipe (40).

Preferably, the breathing support device further includes respirators (43) to cover ears and noses of users, the respirators (43) being provided in ends of the intake/exhaust pipes (42).

According to another aspect of the invention, there is provided a breathing support device including: an inbreathing connection pipe (20a); an inbreathing induction pipe (30a) connected to one end of the inbreathing connection pipe (20a); an inbreathing supply pipe (40a) connected to the other end of the inbreathing connection pipe (20a); an outbreathing connection pipe (20b); an outbreathing discharge pipe (30b) connected to one end of the outbreathing connection pipe (20n); and an outbreathing exhaust pipe (40b) connected to the other end of the outbreathing connection pipe (20b), wherein openings provided in the bottoms of the inbreathing induction pipe (30a) and the outbreathing discharge pipe (30b) are respectively combined with meshes (31), each having a plurality of apertures (31a), which are fixed by respective fastening pieces (32), and cover plates (41a and 41b) are respectively combined to openings of the inbreathing supply pipe (40a) and the outbreathing exhaust pipe (40b) such that the interiors of the inbreathing supply pipe (40a) and the outbreathing exhaust pipe 40b are respectively sealed by the cover plates (41a and 41b), one or more inbreathing pipes (42a) are combined to the cover plate (41a) of the inbreathing supply pipe (40a) such that the interior of the inbreathing supply pipe (40a) communicates to the inbreathing pipes (42a), one or more outbreathing pipes (42b) are combined to the cover plate (41b) of the outbreathing exhaust pipe (40b) such that the interior of the outbreathing exhaust pipe (40b) communicates to the outbreathing pipes (42b), and an inbreathing sealing member (45a) to be regulated by a regulating projection (44a) is provided in the inside of each of the inbreathing pipes (42a) and is opened forwardly for inbreathing, while an outbreathing sealing member (45b) to be regulated by a regulating projection (44b) is provided in the inside of each of the outbreathing pipes (42b) and is opened backwardly for outbreathing.

Preferably, the breathing support device further including respirators (43) respectively provided in ends of the inbreathing and outbreathing pipes (42a and 42b) in a one-to-one correspondence relationship between the inbreathing and outbreathing pipes (42a and 42b).

According to the aspects of the present invention, it is possible to provide a breathing support device for protecting persons confined in a building where toxic fumes are emitted in a disaster such as a fire against toxic fumes, which is capable of supplying fresh air into any internal regions of a building to minimize life accidents due to toxic fumes, thereby allowing the persons to keep conscious to the utmost limit until they are rescued from the toxic fumes. In addition, the breading support device can be useful for maintenance of manholes and tanks in a risk of toxic fumes, underwater operation, etc.

### BRIEF DESCRIPTION OF THE DRAWINGS

The above and/or other aspects and advantages of the present invention will become apparent and more readily appreciated from the following description of the embodiments, taken in conjunction with the accompanying drawings of which:

FIG. 1 is a reference perspective view showing a breathing support device according to a preferred embodiment of the present invention;

FIG. 2 is a reference sectional view showing an internal structure of the breathing support device according to the preferred embodiment of the present invention;

FIG. 3 is a reference sectional view showing a breathing support device according to another embodiment of the present invention;

FIGS. 4A to 4D are reference views showing various forms of a supply pipe according to embodiments of the present invention;

FIG. 5 is a perspective view showing a breathing support device according to still another embodiment of the present invention;

FIGS. 6A and 6B are reference sectional views showing installation and operation of an inhalation pipe according to still another embodiment of the present invention;

FIGS. 7A and 7B are reference sectional views showing installation and operation of an exhalation pipe according to still another embodiment of the present invention; and

FIG. 8 is a reference view showing aspects of use of a breathing support device of the present invention.

### DETAILED DESCRIPTION OF THE PREFERRED EMBODIMENT

### First Embodiment

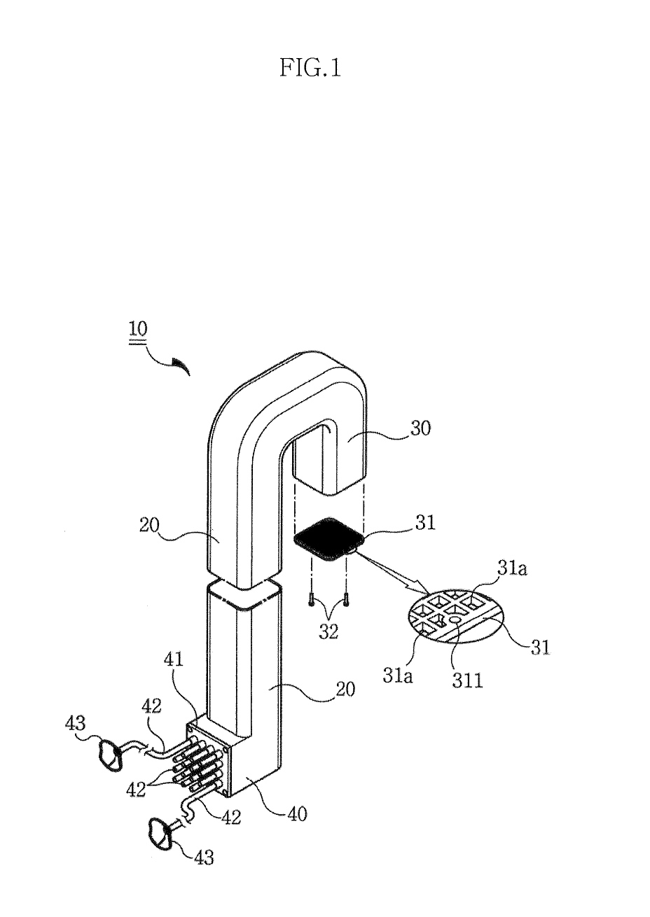

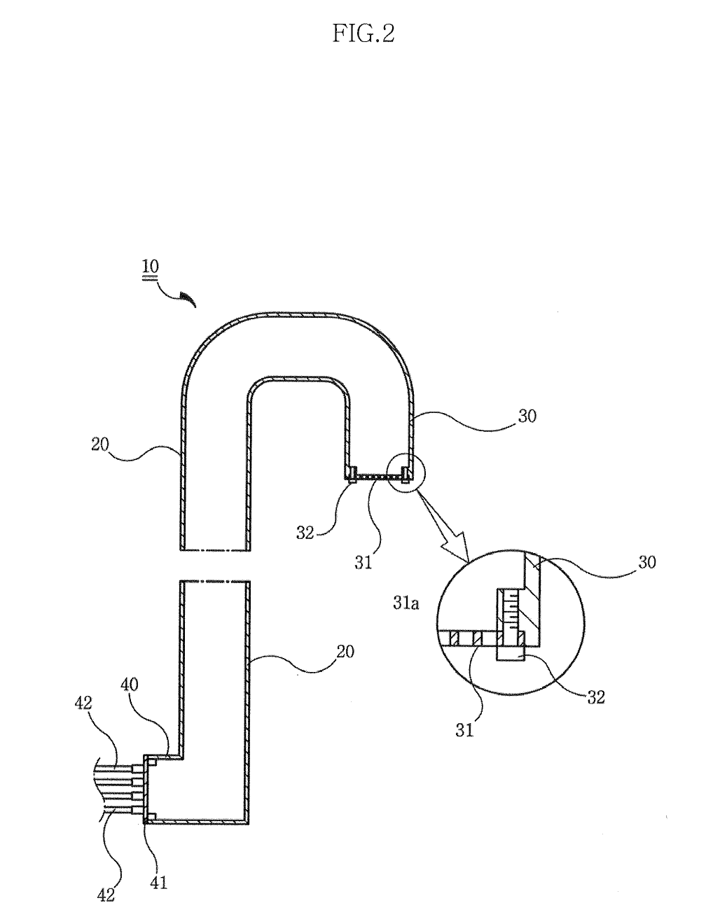

FIG. 1 is a reference perspective view showing a breathing support device 10 according to a first preferred embodiment of the present invention and FIG. 2 is a reference sectional view showing an internal structure of the breathing support device 10 shown in FIG. 1. As shown in the figures, the breathing support device 10 includes a connection pipe 20 having any length, an induction pipe 30 formed above the connection pipe 20, and a supply pipe 40 formed below the connection pipe 20.

The induction pipe 30 integrated with the connection pipe 20 extends downward in the form of “∩” with its opening directing downward to prevent rainwater from being introduced into the connection pipe 20. The opening provided in the bottom of the induction pipe 30 is combined with a mesh 31 having a plurality of apertures 31a which is then fixed by a fastening piece 32 to prevent external alien substances from being introduced into the connection pipe 20. Although the induction pipe 30 is formed to extend downward in the form of “∩” above the connection pipe 20 as shown in FIG. 1, one or more induction pipes 30 may be formed in the side, bottom and/or top of the connection pipe 20 as circumstances demand. In this embodiment, the induction pipe 30 may have other various forms such as “ [symbol] ”, “ [symbol] ”, “ [symbol] ” in addition to “ [symbol] ”.

The supply pipe 40 extends in any lateral side with it integrated with the connection pipe 20. A cover plate 41 is combined to an opening of the supply pipe 40 such that the interior of the supply pipe 40 is sealed by the cover plate 41. One or more intake/exhaust pipes 42 are combined to the cover plate 41 such that the intake/exhaust pipes 42 communicate to the interior of the supply pipe 40.

Preferably, respirators 43 to cover ears and noses of users are further provided in ends of the intake/exhaust pipes 42. The number of intake/exhaust pipes 42 provided in the cover plate 41 and the number of respirators 43 provided in ends of the intake/exhaust pipes 42 may be increased/decreased depending on the number of users.

In this embodiment, the connection pipe 20 connects the induction pipe 30 and the supply pipe 40 and can have a tubular body in the form of a circle or a polygon or may be bent into any shape depending on conditions. Length of the connection pipe 20 may be set depending on height and floor number of a building. However, it should be understood that any modifications of the shape, length and so on of the connection pipe 20 are within the spirit and scope of the invention.

The connection pipe 20 may have a flexible tubular body within the spirit and scope of the invention and the induction pipe 30 and the supply pipe 40 may be provided in the top and bottom of the connection pipe 20, respectively. With this configuration, since the shape of the connection pipe 20 can be changed in any manner, the breathing support device 10 can be installed without regard to shape of a building.

### Second Embodiment

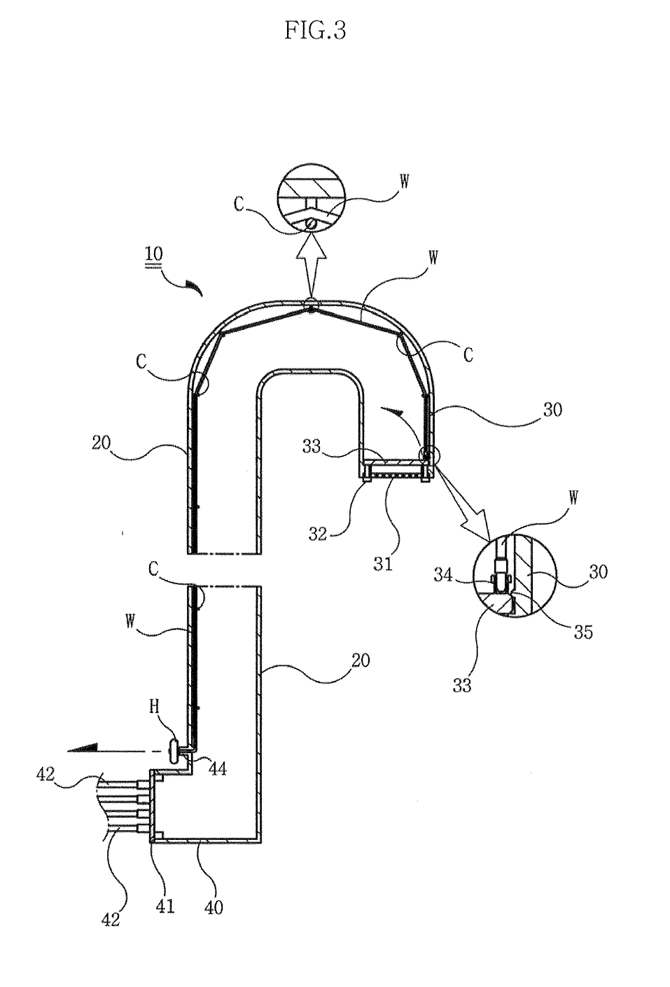

FIG. 3 is a reference sectional view showing a breathing support device 10 according to a second embodiment of the present invention. As shown in the figure, an induction pipe 30 and a supply pipe 40 are formed in the top and bottom of a connection pipe 20, respectively, an opening in the bottom of the induction pipe 30 is combined with a mesh 31 having a plurality of apertures 31a which is then fixed by a fastening piece 32, and one or more intake/exhaust pipes 42 are combined to a cover plate 41 combined to an opening of the supply pipe 40. A sealing plate 33 is rested on a step formed on the top of the opening of the induction pipe 30 such that the sealing plate 33 is regulated by a hemispherical regulating projection 35, clamps C are formed with an interval within the connection pipe 20, an outlet hole 44 is formed in one side of the lower part of the connection pipe 20, a wire W is combined to a fastening bracket 34 provided in the sealing plate 33 such that the wire W is drawn through the outlet hole 44 via each of the clamps C provided within the connection pipe 20, and an end of the wire W drawn through the outlet hole 44 is combined to a handle H such that one side of the sealing plate 33 is lift up by the wire W when the handle H is pulled.

Preferably, respirators 43 to cover ears and noses of users are further provided in ends of the intake/exhaust pipes 42. The number of intake/exhaust pipes 42 provided in the cover plate 41 and the number of respirators 43 provided in ends of the intake/exhaust pipes 42 may be increased/decreased depending on the number of users.

With this configuration, it is possible to prevent fine alien substances from being introduced into the connection pipe 20 since the interior of the induction pipe 30 can be usually isolated from its exterior by the sealing plate 33 and it is also possible to supply fresh air for users since the handle H can be pulled to communicate the connection pipe 20 to the air in the event of a disaster.

At this time, by providing a rotating body such as a pulley in each of the clamps C through which the wire W passes, the wire W can be smoothly pulled by even a small force.

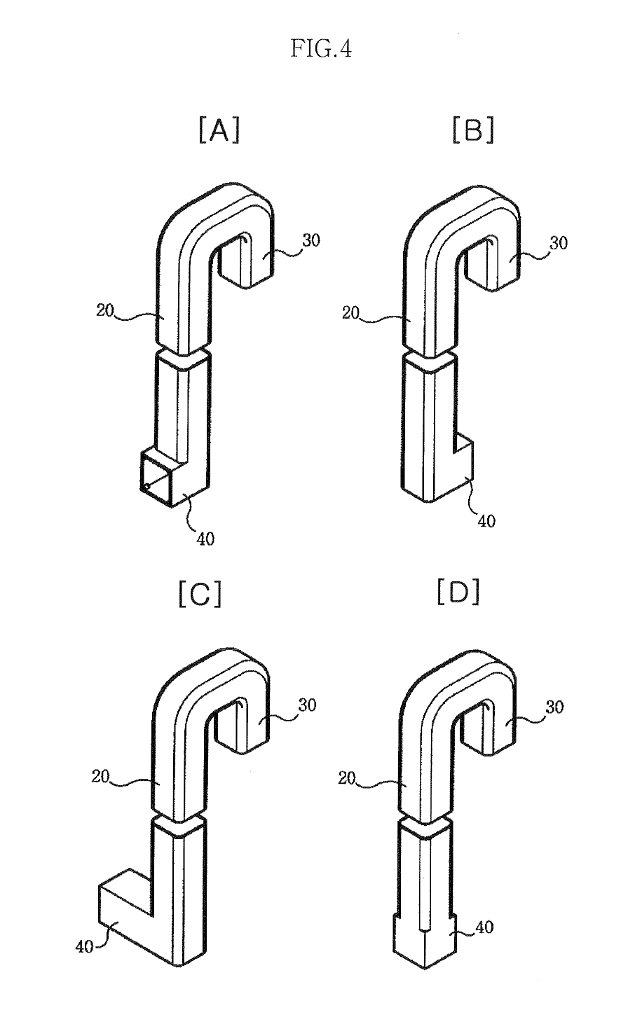

FIGS. 4A to 4D are reference views showing various forms of the supply pipe 40 of the breathing support device 10 of the present invention.

### Third Embodiment

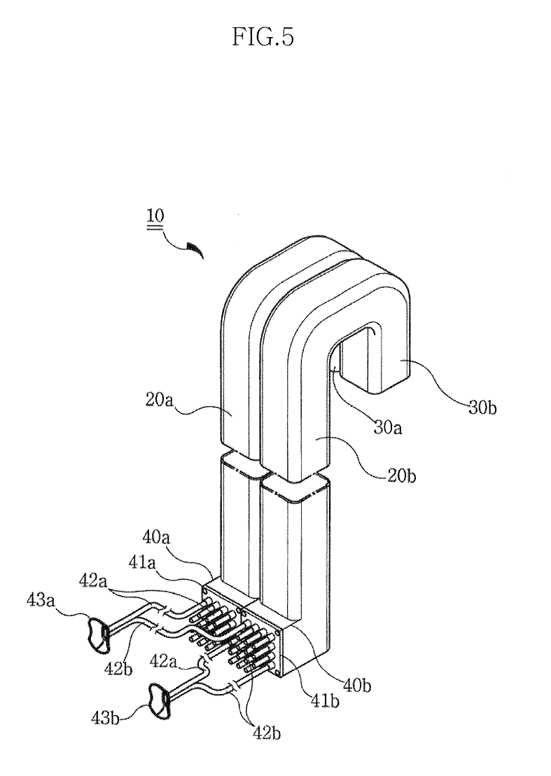

FIG. 5 is a perspective view showing a breathing support device 10 according to a third embodiment of the present invention. As shown in the figure, the breathing support device 10 includes an inbreathing connection pipe 20a, an inbreathing induction pipe 30a formed above the inbreathing connection pipe 20a, an inbreathing supply pipe 40a formed below the inbreathing connection pipe 20a, an outbreathing connection pipe 20b, an outbreathing discharge pipe 30b formed above the outbreathing connection pipe 20b, an outbreathing exhaust pipe 40b formed below the outbreathing connection pipe 20b, such that the supply of inbreathing and the discharge of outbreathing are performed in different regions, thereby preventing many users from be inconvenient in respiration due to carbon dioxide generated in the course of outbreathing, which will be described in more detail below.

The inbreathing induction pipe 30a formed above the inbreathing connection pipe 20a extends downward in the form of “ [symbol] ” with its opening directing downward to prevent rainwater from being introduced into the inbreathing connection pipe 20a. An opening provided in the bottom of the inbreathing induction pipe 30a is combined with a mesh 31 having a plurality of apertures 31a which is then fixed by a fastening piece 32 to prevent external alien substances from being introduced into the inbreathing connection pipe 20a.

The outbreathing discharge pipe 30b formed above the outbreathing connection pipe 20b extends downward in the form of “ [symbol] ” with its opening directing downward to prevent rainwater from being introduced into the outbreathing connection pipe 20b. An opening provided in the bottom of the outbreathing discharge pipe 30b is combined with a mesh 31 having a plurality of apertures 31a which is then fixed by a fastening piece 32 to prevent external alien substances from being introduced into the outbreathing connection pipe 20b.

Although the inbreathing induction pipe 30a and the outbreathing discharge pipe 30b are formed to extend downward in the form of “ [symbol] ” above the inbreathing connection pipe 20a and the outbreathing connection pipe 20b, respectively, as shown in FIG. 1, one or more inbreathing induction pipe 30a and one or more outbreathing discharge pipe 30b may be formed in the side, bottom and/or top of the inbreathing connection pipe 20a and outbreathing connection pipe 20b as circumstances demand. In this embodiment, the inbreathing induction pipe 30a and the outbreathing discharge pipe 30b may have other various forms such as “ [symbol] ”, “ [symbol] ”, “ [symbol] ” in addition to “ [symbol] ”.

Cover plates 41a and 41b are combined to openings of the inbreathing supply pipe 40a and outbreathing exhaust pipe 40b such that the interiors of the inbreathing supply pipe 40a and outbreathing exhaust pipe 40b are sealed by the cover plates 41a and 41b, respectively. One or more inbreathing pipes 42a are combined to the cover plate 41a of the inbreathing supply pipe 40a such that the interior of the inbreathing supply pipe 40a communicates to the inbreathing pipes 42a, and one or more outbreathing pipes 42b are combined to the cover plate 41b of the outbreathing exhaust pipe 40b such that the interior of the outbreathing exhaust pipe 40b communicates to the outbreathing pipes 42b.

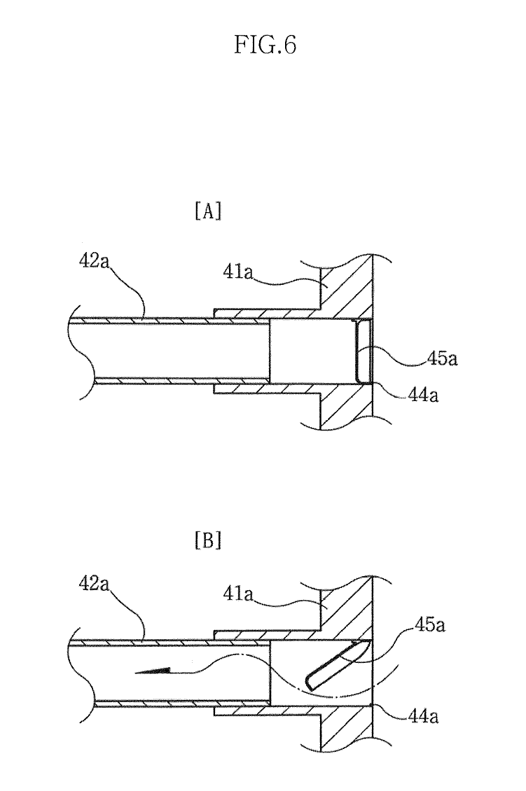

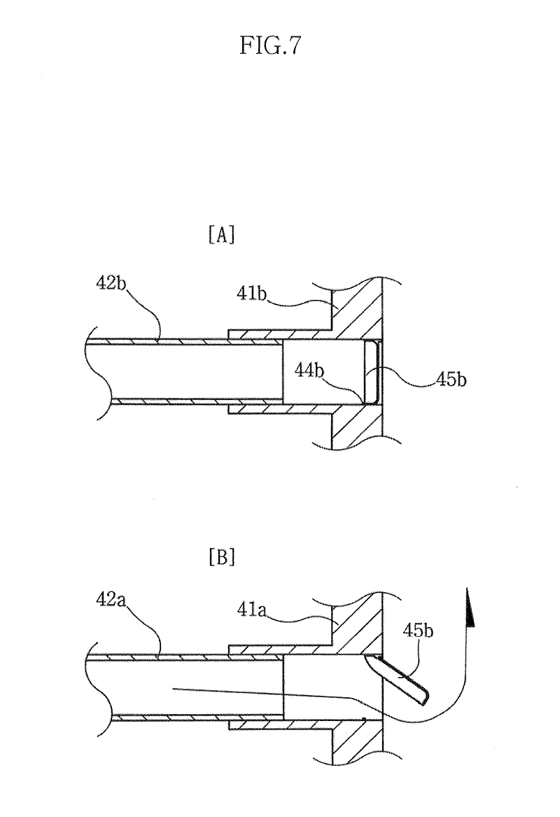

As shown in FIGS. 6 and 7, an inbreathing sealing member 45a to be regulated by a regulating projection 44a is provided in the inside of the inbreathing pipe 42a and can be opened forwardly for inbreathing, while an outbreathing sealing member 45b to be regulated by a regulating projection 44b is provided in the inside of the outbreathing pipe 42b and can be opened backwardly for outbreathing. The inbreathing sealing member 45a and the outbreathing sealing member 45b are made of soft synthetic resin having excellent elasticity and restoration and may be implemented with typical sealing members (regulating members) used to allow unidirectional movement of air, detailed explanation of which will be omitted for the purpose of brevity.

The inbreathing pipes 42a and the outbreathing pipes 42b combined to the respective cover plates 41a and 41b have a one-to-one correspondence relationship.

Preferably, respirators 43a and 43b are further provided in ends of the inbreathing and outbreathing pipes 42a and 42b, respectively. The number of inbreathing and outbreathing pipes 42a and 42b combined to the respective cover plates 41a and 41b and the number of respirators 43a and 43b provided in their ends may be increased/decreased depending on the number of users.

As an alternative, the inbreathing pipes 42a may be formed to be longer than the outbreathing pipes 42b to allow users to hold the inbreathing pipes 42a with ends of the outbreathing pipes 42b exposed to the external. This allows the users to keep breathing in such a manner that the users inhale fresh air through the inbreathing pipes 42a with exhaled air discharged to the external through the exposed ends of the outbreathing pipes 42b.

<Operation of the Invention>

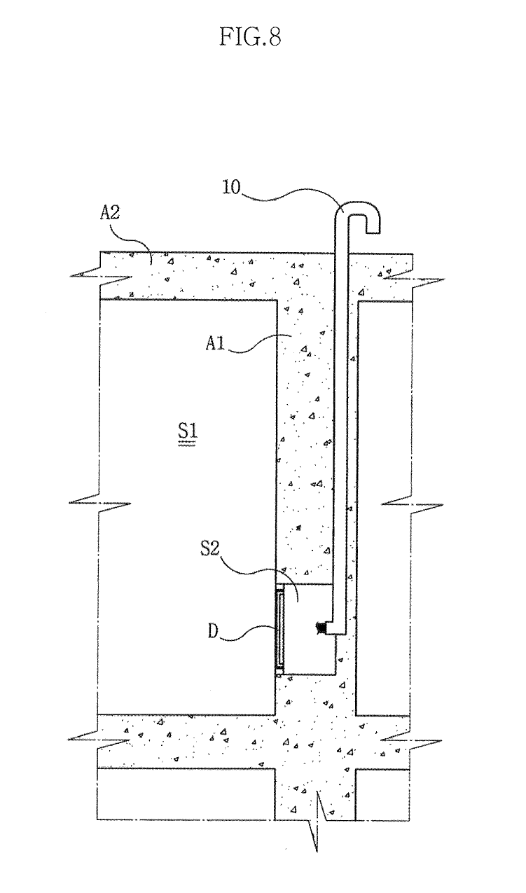

FIG. 8 is a reference view showing aspects of use of a breathing support device of the present invention. As shown in the figure, a connection pipe 20 is buried in a side wall A1 of a place such as a shelter S1 provided in a building, with the top of the connection pipe 20 passing through a roof slab A2 to locate an induction pipe 30, and a hollow storage space S2 having an openable/closable door D is formed in the side wall A1 to store a supply pipe 40 provided in the bottom of the connection pipe 20. This configuration allows users to keep breathing with fresh air using respirators 43 provided in the storage space S2 in the event of a disaster such as a fire.

In the present invention, since the connection pipe 20 and the supply pipe 40 have their respective considerable diameter and the branching intake/exhaust pipes 42 branching from the supply pipe 40 are used to keep the user breathing, the users can have moderate respiration.

Although it has been illustrated in the above that the breathing support device 10 of the present invention is used for a disaster occurring in a building, the breathing support device 10 may be useful for maintenance of manholes and tanks in a risk of toxic fumes, underwater operation, drift works in a risk of dusts and toxic fumes, painting operations using air brushes, etc.

While the present invention has been particularly shown and described with reference to exemplary embodiments thereof, it will be understood by those skilled in the art that various changes in form and details may be made therein without departing from the spirit and scope of the present invention. The exemplary embodiments are provided for the purpose of illustrating the invention, not in a limitative sense. Thus, it is intended that the present invention covers the modifications and variations of this invention provided they come within the scope of the appended claims and their equivalents.

What is claimed is:

1. A breathing support device (10) comprising:
a connection pipe (20) having any length;
an induction pipe (30) connected to one end of the connection pipe (20); and
a supply pipe (40) connected to the other end of the connection pipe (20),
wherein an opening provided in the bottom of the induction pipe (30) is combined with a mesh (31) having a plurality of apertures (31a) which is fixed by a fastening piece (32), and
a cover plate (41) is combined to an opening of the supply pipe (40) such that the interior of the supply pipe (40) is sealed by the cover plate (41) and one or more intake/exhaust pipes (42) are combined to the cover plate (41) such that the intake/exhaust pipes (42) communicate to the interior of the supply pipe (40).

2. The breathing support device according to claim 1, further comprising respirators (43) to cover ears and noses of users, the respirators (43) being provided in ends of the intake/exhaust pipes (42).

3. The breathing support device according to claim 1, wherein a sealing plate (33) is rested on a step formed on the top of the opening of the induction pipe (30), an inbreathing induction pipe (30a) or an outbreathing discharge pipe (30b) such that the sealing plate (33) is regulated by a hemispherical regulating projection (35), clamps (C) are formed with an interval within the connection pipe (20), an inbreathing connection pipe (20a) or an outbreathing connection pipe (20b), an outlet hole (44) is formed in one side of the lower part of the connection pipe (20), the inbreathing connection pipe (20a) or the outbreathing connection pipe (20b), a wire (W) is combined to a fastening bracket (34) provided in the sealing plate (33) such that the wire W is drawn through the outlet hole (44) via each of the clamps (C) provided within the connection pipe 20, the inbreathing connection pipe (20a) or the outbreathing connection pipe (20b), and a handle (H) is combined to an end of the wire W drawn through the outlet hole (44).

4. The breathing support device according to claim 1, wherein the connection pipe (20), an inbreathing connection pipe (20a) or an outbreathing connection pipe (20b) has a flexible tubular body, and the induction pipe (30), an inbreathing induction pipe 30a or an outbreathing discharge pipe (30b) and the supply pipe (40), an inbreathing supply pipe 40a or an outbreathing exhaust pipe 40b are provided in the top and bottom of the connection pipe 20, the inbreathing connection pipe (20a) or the outbreathing connection pipe (20b), respectively.

5. The breathing support device according to claim 2, wherein a sealing plate (33) is rested on a step formed on the top of the opening of the induction pipe (30), an inbreathing induction pipe (30a) or an outbreathing discharge pipe (30b) such that the sealing plate (33) is regulated by a hemispherical regulating projection (35), clamps (C) are formed with an interval within the connection pipe (20), an inbreathing connection pipe (20a) or an outbreathing connection pipe (20b), an outlet hole (44) is formed in one side of the lower part of the connection pipe (20), the inbreathing connection pipe (20a) or the outbreathing connection pipe (20b), a wire (W) is combined to a fastening bracket (34) provided in the sealing plate (33) such that the wire W is drawn through the outlet hole (44) via each of the clamps (C) provided within the connection pipe 20, the inbreathing connection pipe (20a) or the outbreathing connection pipe (20b), and a handle (H) is combined to an end of the wire W drawn through the outlet hole (44).

6. The breathing support device according to claim 2, wherein the connection pipe (20), an inbreathing connection pipe (20a) or an outbreathing connection pipe (20b) has a flexible tubular body, and the induction pipe (30), an inbreathing induction pipe (30a) or an outbreathing discharge pipe (30b) and the supply pipe (40), an inbreathing supply pipe (40a) or an outbreathing exhaust pipe (40b) are provided in the top and bottom of the connection pipe (20), the inbreathing connection pipe (20a) or the outbreathing connection pipe (20b), respectively.

7. A breathing support device (10) comprising:
an inbreathing connection pipe (20a);
an inbreathing induction pipe (30a) connected to one end of the inbreathing connection pipe (20a);
an inbreathing supply pipe (40a) connected to the other end of the inbreathing connection pipe (20a);
an outbreathing connection pipe (20b);
an outbreathing discharge pipe (30b) connected to one end of the outbreathing connection pipe (20n); and
an outbreathing exhaust pipe (40b) connected to the other end of the outbreathing connection pipe (20b),
wherein openings provided in the bottoms of the inbreathing induction pipe (30a) and the outbreathing discharge pipe (30b) are respectively combined with meshes (31), each having a plurality of apertures (31a), which are fixed by respective fastening pieces (32), and
cover plates (41a and 41b) are respectively combined to openings of the inbreathing supply pipe (40a) and the outbreathing exhaust pipe (40b) such that the interiors of the inbreathing supply pipe (40a) and the outbreathing exhaust pipe 40b are respectively sealed by the cover plates (41a and 41b), one or more inbreathing pipes (42a) are combined to the cover plate (41a) of the inbreathing supply pipe (40a) such that the interior of the inbreathing supply pipe (40a) communicates to the inbreathing pipes (42a), one or more outbreathing pipes (42b) are combined to the cover plate (41b) of the outbreathing exhaust pipe (40b) such that the interior of the outbreathing exhaust pipe (40b) communicates to the outbreathing pipes (42b), and an inbreathing sealing member (45a) to be regulated by a regulating projection (44a) is provided in the inside of each of the inbreathing pipes (42a) and is opened forwardly for inbreathing, while an outbreathing sealing member (45b) to be regulated by a regulating projection (44b) is provided in the inside of each of the outbreathing pipes (42b) and is opened backwardly for outbreathing.

8. The breathing support device according to claim 7, further comprising respirators (43) respectively provided in ends of the inbreathing and outbreathing pipes (42a and 42b) in a one-to-one correspondence relationship between the inbreathing and outbreathing pipes (42a and 42b).

9. The breathing support device according to claim 7, wherein a sealing plate (33) is rested on a step formed on the top of the opening of the inbreathing induction pipe (30a) or the outbreathing discharge pipe (30b) such that the sealing plate (33) is regulated by a hemispherical regulating projection (35), clamps (C) are formed with an interval within the inbreathing connection pipe (20a) or the outbreathing connection pipe (20b), an outlet hole (44) is formed in one side of the lower part of the inbreathing connection pipe (20a) or the outbreathing connection pipe (20b), a wire (W) is combined to a fastening bracket (34) provided in the sealing plate (33) such that the wire W is drawn through the outlet hole (44) via each of the clamps (C) provided within the inbreathing connection pipe (20a) or the outbreathing connection pipe (20b), and a handle (H) is combined to an end of the wire W drawn through the outlet hole (44).

10. The breathing support device according to claim 7, wherein the inbreathing connection pipe (20a) or the outbreathing connection pipe (20b) has a flexible tubular body, and the inbreathing induction pipe 30a or the outbreathing discharge pipe (30b) and the inbreathing supply pipe (40a) or the outbreathing exhaust pipe (40b) are provided in the top and bottom of the inbreathing connection pipe (20a) or the outbreathing connection pipe (20b), respectively.

11. The breathing support device according to claim 8, wherein a sealing plate (33) is rested on a step formed on the top of the opening of the inbreathing induction pipe (30a) or the outbreathing discharge pipe (30b) such that the sealing plate (33) is regulated by a hemispherical regulating projection (35), clamps (C) are formed with an interval within the inbreathing connection pipe (20a) or the outbreathing connection pipe (20b), an outlet hole (44) is formed in one side of the lower part of the inbreathing connection pipe (20a) or the outbreathing connection pipe (20b), a wire (W) is combined to a fastening bracket (34) provided in the sealing plate (33) such that the wire W is drawn through the outlet hole (44) via each of the clamps (C) provided within the inbreathing connection pipe (20a) or the outbreathing connection pipe (20b), and a handle (H) is combined to an end of the wire W drawn through the outlet hole (44).

12. The breathing support device according to claim 8, wherein the inbreathing connection pipe (20a) or the outbreathing connection pipe (20b) has a flexible tubular body, and the inbreathing induction pipe (30a) or the outbreathing discharge pipe (30b) and the inbreathing supply pipe (40a) or the outbreathing exhaust pipe (40b) are provided in the top and bottom of the inbreathing connection pipe (20a) or the outbreathing connection pipe (20b), respectively.
